SEQUENCE LISTING (4 sequences)

SEQ ID NO: 1 - protein, 274 aa, synthetic construct
AWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 2 - protein, 274 aa, synthetic construct
AWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLAQTSYLCVCAEGFAPIPHEPHRCQMFCAQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 3 - protein, 40 aa, synthetic construct
QMFCNQTACPADCDPNTQASCECPEGYILDDGFICTDIDE

SEQ ID NO: 4 - protein, 19 aa, synthetic construct
ECPEGYILDDGFICTDIDE

(19) United States
(12) Patent Application Publication
(10) Pub. No.: US 2024/0269240 A1
(43) Pub. Date: Aug. 15, 2024

(54) COMPOSITIONS AND METHODS FOR TREATING HAIR LOSS OR FACILITATING HAIR GROWTH

(71) Applicant: BLUE BLOOD BIOTECH CORPORATION, Taipei City (TW)

(72) Inventors: Hua-Lin WU, Taipei City (TW); Chien-Chen LIN, Taipei City (TW); Jiun-Yan DING, Taipei City (TW)

(21) Appl. No.: 18/169,575

(22) Filed: Feb. 15, 2023

(51) Int. Cl. A61K 38/36; A61P 17/14

(52) U.S. Cl. CPC A61K 38/366; A61P 17/14

(57) ABSTRACT

Provided is a method for treating or preventing hair loss or facilitating hair growth or regrowth. More particularly, it relates to a composition for preventing or treating hair loss and/or facilitating hair growth on the scalp and/or skin of a subject in need thereof. The composition includes an effective amount of a polypeptide or a nucleic acid molecule encoding the polypeptide, and the polypeptide comprises an amino acid sequence having an EGF-like domain of thrombomodulin or a conservative variant thereof.

### SEQUENCE LISTING

Pursuant to 37 CFR § 1.831-835, the instant application contains a computer readable Sequence Listing which has been submitted electronically in XML format and is hereby incorporated by reference in its entirety. Said XML format file, created on Feb. 13, 2023, is named 211140US-Sequence Listing-20230213 and is 4.75 kb in size.

### TECHNICAL FIELD

The present disclosure relates to hair maintenance and enrichment, particularly to preventing and/or slowing down hair loss and facilitating hair growth and/or regrowth on the scalp and/or skin of a mammal.

### BACKGROUND ART

Hair loss, also called alopecia, is caused by an interruption in the body's cycle of hair production. In mammals, hair goes through three cycles:
(i) Anagen: an active stage, during which the hair follicle penetrates deep into the dermis with the cells of the bulb dividing rapidly and differentiating to form the hair,
(ii) Catagen: a regressive stage, during which the follicle regresses upwards through the dermis and hair growth ceases,
(iii) Telogen: a resting stage, in which the regressed follicle contains a small secondary germ with an underlying ball of tightly packed dermal papilla cells.

The initiation of a new anagen stage is revealed by rapid proliferation in the germ, expansion of the dermal papilla and elaboration of basement membrane components. The hair cycle is then repeated many times until, as a consequence of the onset of male pattern baldness, most of the hair follicles spend an increasing proportion of their time in the telogen stage, and the hairs produced become finer, shorter, and less visible; this is known as terminal to vellus transformation.

Hair loss can occur anywhere on the body, but most commonly affects the scalp. On average, the human scalp has 100,000 hairs that cycle through periods of growing, resting, falling out, and regenerating. Signs and symptoms of hair loss on the scalp may include: gradual thinning on top of the head, circular or patchy bald spots, sudden loosening of hair, and patches of scaling that spread over the scalp. In general, excessive hair loss can be the result of heredity, hormonal changes, medical conditions (such as cancer chemotherapies) or a normal part of aging.

As the population ages and lives a stressful life in modern society, a growing number of people are under the threat of hair loss. It is estimated that 40% of 35-year-old males in the United States will have some noticeable hair loss. This condition gets worse as men get older. By age 60, it is estimated that 65% of adult males in the United States will suffer from noticeable hair loss. Women also suffer from hair loss. Female hair loss, unlike in men, typically involves noticeable thinning all over the head and the hairline. The American Academy of Dermatology reports that 40% of women have noticeable hair loss by the age of 40. Although excessive hair loss on the scalp is mostly benign medically, it has great cosmetic impacts and can bring tremendous emotional and psychosocial pressure to individuals and may deeply affect a person's self-confidence and social life. For instance, hair loss may not be seen as normal or acceptable for women due in part to social norms that may pressure women to look their best. Even a slight thinning of hair, if noticeable, may be undesirable to a woman's self-image and how others may judge her.

Current treatments for hair loss and/or baldness include medications and hair transplantation: however, the wide-spread dissatisfactions indicate they are not enough to propel the hair of a relatively regressive and/or resting status toward an active stage, probably due to the diversity of reasons for hair loss and the complexity of the mechanism of hair growth.

Medications such as MINOXIDIL® (Rogains). FINASTERIDE® (Propecia) and DUTASTERIDER (Avodart) are an approved treatment for hair loss. However, these medications have shortcomings because, to be effective, the patient is required to take the medication daily. Further, the medication will work in the long term only if the patient continues to take the medication. Any discontinuance is likely to result in the loss of any hair gained. Moreover, some of these medications are not approved for use by women because of safety concerns.

Invasive hair loss treatment available includes surgical procedures such as follicle unit transplantation, scalp reduction, or scalp flap. These surgical procedures have a risk of possible skin necrosis or keloid scar tissue formation at the donor region. These surgical procedures also often produce unsatisfactory results such as unnatural looking hair growth due to the direction of hair growth and limitations on the number of hair follicles that can be safely transferred (less than 4000 hair follicles can be transferred every procedure with a maximum of 3 procedures in a patient's entire life). Such surgical treatment is also inconvenient for patients because it often requires 3-4 hours of operation, post-operative care, and disruption of normal daily living immediately after the surgery for approximately 2 weeks (even without complications).

Currently, there are also many different types of injection methods, such as platelet rich plasma (PRP)-alone injection used for hair loss treatment. A wealth of literature has already considered the use of PRP in sports medicine, orthopedic surgery, dental surgery, and a number of other medical and surgical specialties to enhance tissue repair and healing after surgical procedures or injury. However, the results of these injection methods vary widely. The existing injection method treatment, while restoring hair to some individuals, lacks the consistency to make it work for significant other populations suffering from hair loss.

While various attempts have been made as explained above, current treatments for hair loss have not necessarily had sufficient hair care actions such as prevention of hair loss and promotion of hair growth in any area of the scalps. Finasteride and minoxidil treatments claimed to work on both the vertex area and the frontal area, but are most successful in only the vertex area. In addition, most baldness treatment takes a long period, such as four to six months, before apparently showing the treatment effect. Possibly, the only means which has met with partial success for growing hair on the bald or balding human head is by transplantation of hair to the bald areas. This is usually a painful operation and is not always successful. Furthermore, it is immediately apparent to the casual observer that the subject has received a hair transplant and it may take many months or even years before hair regrowth, following this operation, assumes an appearance which resembles that of the original naturally growing hair.

It is therefore desirable to provide a novel composition effective for preventing hair loss and promoting hair growth and/or regrowth on the scalp and/or skin.

### SUMMARY OF THE INVENTION

In view of the foregoing, the present disclosure provides a composition that has a hair loss preventing effect, hair growth promoting effect, and hair care effect in humans and other mammals and is useful as a pharmaceutical, quasi-pharmaceutical, or cosmetic composition for hair care. The composition comprises an effective amount of a polypeptide comprising an amino acid sequence having an EGF-like domain of thrombomodulin or a conservative variant thereof.

Thrombomodulin is an anticoagulant, endothelial-cell-membrane glycoprotein. Recombinant thrombomodulin proteins, TMD2 (containing EGF-like structures) and TMD23 (containing TMD2 and a serine-threonine rich domain), exhibit some activity of cell behavior.

In at least one embedment of the present disclosure, the composition comprises an effective amount of a recombinant polypeptide comprising an amino acid sequence having an EGF-like domain of thrombomodulin or a conservative variant thereof. In other embodiments, the polypeptide is a recombinant polypeptide comprising an amino acid sequence of EGF-like structures of thrombomodulin operably linked to a serine-threonine rich domain of the thrombomodulin or a conservative variant thereof. In at least one embodiment, the number of the EGF-like structures is one to six. In some embodiments, the number of the EGF-like structures is six.

The present invention provides a method for preventing or treating hair loss or facilitating hair growth or regrowth, comprising administering to a subject in need thereof with a therapeutically effective amount of a polypeptide or a nucleic acid molecule encoding the polypeptide, and the polypeptide comprises an amino acid sequence having EGF-like domain of thrombomodulin or a conservative variant thereof.

In the preferred embodiment, the polypeptide is a recombinant polypeptide comprising an amino acid sequence of EGF-like structures of the thrombomodulin operably linked to a serine-threonine rich domain of the thrombomodulin or the conservative variant thereof. In some embodiments, the number of the EGF-like structures is six. In further embodiments, the recombinant polypeptide essentially consists of an amino acid sequence of six EGF-like structures of the thrombomodulin operably linked to a serine-threonine rich domain of the thrombomodulin or the conservative variant thereof.

In at least one embodiment, the polypeptide is at least 35% identical to TMD23 having the amino acid sequence of SEQ ID NO: 1 as follows:

AWDCSVENGGCEHACNAIPGAPRCQCPAGAALQADGRSCTASATQSCND
LCEHFCVPNPDQPGSYSCMCETGYRLAADQHRCEDVDDCILEPSPCPQR
CVNTQGGFECHCYPNYDLVDGECVEPVDPCFRANCEYQCQPLNQTSYLC
VCAEGFAPIPHEPHRCQMFCNQTACPADCDPNTQASCECPEGYILDDGF
ICTDIDECENGGFCSGVCHNLPGTFECICGPDSALARHIGTDCDSGKVD
GGDSGSGEPPPSPTPGSTLTPPAVGLVHS

In some embodiments, the polypeptide essentially consists of TMD23. In some embodiments, the polypeptide is TMD23.

In at least one embodiment, the polypeptide essentially consists of BB101 having the amino acid sequence of SEQ ID NO: 2 as follows:

AWDCSVENGGCEHACNAIPGAPRCQCPAGAALQADGRSCTASATQSCND
LCEHFCVPNPDQPGSYSCMCETGYRLAADQHRCEDVDDCILEPSPCPQR
CVNTQGGFECHCYPNYDLVDGECVEPVDPCFRANCEYQCOPLAQTSYLC
VCAEGFAPIPHEPHRCQMFCAQTACPADCDPNTQASCECPEGYILDDGF
ICTDIDECENGGFCSGVCHNLPGTFECICGPDSALARHIGTDCDSGKVD
GGDSGSGEPPPSPTPGSTLTPPAVGLVHS

In at least one embodiment, the polypeptide comprises a TME5 structure having the amino acid sequence at least 35% identical to SEQ ID NO: 3 as follows:

QMFCNQTACPADCDPNTQASCECPEGYILDDGFICTDIDE

In some embodiments, the polypeptide comprises a TME5 structure having the amino acid sequence essentially consists of SEQ ID NO: 3. In some embodiments, the TME5 structure has the amino acid sequence of SEQ ID NO: 3.

In at least one embodiment, the polypeptide comprises a TME5C structure having the amino acid sequence at least 35% identical to SEQ ID NO: 4 as follows:

### ECPEGYILDDGFICTDIDE

In some embodiments, the polypeptide comprises a TME5C structure having the amino acid sequence essentially consists of SEQ ID NO: 4. In some embodiments, the TME5C structure has the amino acid sequence of SEQ ID NO: 4.

In at least one embodiment, the nucleic acid molecule is an exogenously introduced polynucleic acids for increasing the expression of the polypeptide in a subject. In some embodiments, the nucleic acid molecule encoding the polypeptide is mRNA.

In further embodiment, the composition further comprises a pharmaceutically or cosmetically acceptable carrier.

In one aspect, the present disclosure provides a method for the treatment of hair loss, comprising administering to a subject in need thereof a composition comprising: a) a recombinant polypeptide comprising an amino acid sequence of EGF-like structures of thrombomodulin operably linked to a serine-threonine rich domain of the thrombomodulin or a conservative variant thereof; and b) a pharmaceutically or cosmetically acceptable carrier.

Common types of hair loss include male- or female-pattern hair loss, alopecia areata, and a thinning of hair known as telogen effluvium. The cause of male-pattern hair loss is a combination of genetics and male hormones: the cause of female pattern hair loss is unclear; the cause of alopecia areata is autoimmune; and the cause of telogen effluvium is typically a physically or psychologically stressful event. Telogen effluvium is also very common following pregnancy.

Less common causes of hair loss without inflammation or scarring include the pulling out of hair, certain medications including chemotherapy, HIV/AIDS, hypothyroidism, and malnutrition including iron deficiency. Causes of hair loss that occur with scarring or inflammation include fungal infection, lupus erythematosus, radiation therapy, and sarcoidosis. Diagnosis of hair loss is partly based on the areas affected.

In at least one embodiment of the present disclosure, hair loss is selected from the group consisting of involutional alopecia, androgenic alopecia, male-pattern hair loss, female-pattern hair loss, alopecia areata, traction alopecia, anagen effluvium, telogen effluvium, cicatricial (scarring) alopecia, hair loss due to infectious diseases, hair loss due to chemotherapy, hair loss due to radiation, hair loss due to hormonal imbalance, hair loss due to medication, hair loss due to chemicals exposure, hair loss due to stress, and any combination thereof.

In at least one embodiment of the present disclosure, hair loss is involutional alopecia.

In at least one embodiment of the present disclosure, hair loss is androgenic alopecia (male- or female-pattern hair loss).

In some embodiments, the composition facilitates hair growth and/or regrowth of the subject. In at least one embodiment of the present disclosure, administration of the composition of the present invention results in one or more of the following: cell growth, hair follicle rejuvenation, invigorated hair follicles, increased hair-shaft size, and new hair growth.

The type of preparation which the composition of the present invention may take is not particularly limited so long as it is a type of preparation which can be applied to the skin, in particular, the scalp. For example, a gel, cream, paste, lotion, spray, suspension, solution, dispersion salve, hydrogel, liquid, emulsion, ointment, a film, an orabase, a powder, liniment, etc. Further, the composition of the present invention may be of any form such as a hair tonic, hair clear lotion, hair care conditioner, hair care gel, hair care mousse and shampoo, etc.

The compositions of the invention comprise a pharmaceutically or cosmetically acceptable carrier to act as a diluent, dispersant, or vehicle for the recombinant thrombomodulin of the present invention, so as to facilitate its distribution when the composition is applied to the skin and/or scalp. Vehicles other than or in addition to water can include liquid or solid emollients, solvents, humectants, thickeners, and powders.

In at least one embodiment, the pharmaceutically or cosmetically acceptable carrier may be from 0.001% to 99.999%, for example, but not limited to. 0.001%. 0.002%. 0.003%. 0.004%, 0.005%, 0.006%, 0.007%, 0.008%, 0.009%, 0.01%, 0.02%. 0.03%, 0.04%, 0.05%, 0.06%. 0.07%, 0.08%, 0.09%, 0.1%, 1%, 2%, 3%, 4%, 5%, 6%, 7%, 8%, 9%. 10%, 15%, 20%. 25%, 30%, 35%, 40%. 45%, 50%, 55%, 60%. 65%, 70%. 75%. 80%, 85%, 90%, 95%, 96%, 97%, 98%, 99%, 99.9999%, by weight of the composition, and can, in the absence of other pharmaceutical or cosmetic adjuncts, form the balance of the composition.

In at least one embodiment of the present application, the compositions may be in the form of aqueous, aqueous/alcoholic or oily solutions; dispersions of the lotion or serum type: anhydrous or lipophilic gels: emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known in the art.

In at least one embodiment of the present application, the compositions of the present disclosure may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. In at least one embodiment, the composition of the present disclosure is locally or systemically delivered to the subject.

In at least one embodiment, the composition of the present disclosure may be administered by a route including subcutaneous injection and intradermal injection. In some embodiments, the composition is administered to the scalp of the subject. In at least one embodiment, the method further comprises applying heat by infrared or hot pack to the skin of the subject. In other embodiments, the composition is administered to the scalp of the subject. In further embodiments, the composition is administered to the subject topically, intradermally, subcutaneously. Preferably, the composition of the present disclosure is administered by intradermal injection.

In at least one embodiment, the method of the present disclosure further comprises applying physical intervention, chemical intervention, or a combination thereof. In some embodiments, the physical intervention may be applied by any device to increase the skin penetration rate. In some embodiments, the physical intervention is selected from the group consisting of needles, microneedles, microneedle patches, micro-needling rollers, micro-needling stamps, Meso Gun, microsurgical device, superficial injection device, needle-free intradermal injection, shaved, temperature elevation, electroporation, ultrasound-mediation, ultrasound-mediated microbubble cavitation, and any combination thereof.

In at least one embodiment, the composition of the present disclosure may be administered topically, intradermally, or subcutaneously onto the target site(s) such as the scalp with various injection tools. In some embodiments, the composition of the present disclosure may be administered to the subject through a transdermal delivery system, comprising needles, microneedles, solid microneedles, coated microneedles, dissolving microneedles, hollow microneedles, microneedle patches to which microneedles are attached, micro-needling (derma) rollers, micro-needling (derma) stamps, Meso Gun, microsurgical device, shave, superficial injection device, electroporation, ultrasound-mediated microbubble cavitation by the application of cavitation-enhancing nano-particles or micro-particles to the skin followed by irradiating that part of the skin with ultrasonic energy, needle-free intradermal injection, and any combination thereof, but the present disclosure is not limited thereto.

In at least one embodiment, the composition of the present invention may be administered topically, intradermally, or subcutaneously onto the target site(s) such as through direct coating or spraying on the skin.

In at least one embodiment of the present disclosure, the polypeptide is at least 35% identical to TMD23 having the amino acid sequence of SEQ ID NO: 1. In some embodiments, the polypeptide of the present disclosure is at least 35% to 99%. 40% to 90%, 55% to 80%, or 50% to 75% identical to the amino acid sequence of SEQ ID NO: 1. In some embodiments, the polypeptide is at least 35%, 40%, 45%, 50%. 55%, 60%, 65%, 70%. 75%, 80%, 85%, 90%, 95%, 96%, 97%, 98%, 99%, or 99.5% identical to the amino acid sequence of SEQ ID NO: 1.

In at least one embodiment, the polypeptide comprises a TME5 structure having the amino acid sequence is at least 35% to 99%, 40% to 90%. 55% to 80%, or 50% to 75% identical to the amino acid sequence of SEQ ID NO: 3. In some embodiments, the polypeptide is at least 35%, 40%, 45%, 50%, 55%, 60%, 65%, 70%, 75%, 80%, 85%, 90%, 95%, 96%, 97%, 98%, 99%, or 99.5% identical to the amino acid sequence of SEQ ID NO: 3.

In at least one embodiment, the polypeptide comprises a TME5C structure having the amino acid sequence is at least 35% to 99%, 40% to 90%. 55% to 80%, or 50% to 75% identical to the amino acid sequence of SEQ ID NO: 4. In some embodiments, the polypeptide is at least 35%, 40%, 45%, 50%, 55%, 60%. 65%, 70%, 75%, 80%, 85%, 90%, 95%, 96%, 97%, 98%, 99%, or 99.5% identical to the amino acid sequence of SEQ ID NO: 4.

The dosage of the composition of the present invention differs depending on age, personal differences, states of the disease, form of the composition, method of application, etc. and cannot be clearly specified. An effective amount of TMD23, BB101 or variants thereof differs depending on age, personal differences, states of the disease of the subjects, form of composition thereof, method of application, and is not particularly limited. Generally, when administered to humans, it can be administered in an amount of 0.05 μg to 1 mg of TMD23, BB101 or variants thereof per cm² of skin surface that is the target of hair growth treatment e.g., about 0.05, 0.1, 0.5, 1, 5, 10, 20, 30, 40, 50, 60, 70, 80, 90, 100, 200, 300, 400, 500, 600, 700, 800, 900, or 1000 μg per cm² of skin surface, but the present disclosure is not limited thereto. In some embodiments, TMD23, BB101 or variants thereof may be used in an amount of 0.05 μg to 100 μg, 0.1 μg to 50 μg, or 10 μg to 200 μg per cm² of skin surface that is the target of hair growth treatment. In further embodiments, TMD23, BB101 or variants thereof may be used at a concentration of 0.05 μg/mL to 200 μg/mL when administered to a subject in need thereof.

The frequency of administration of the composition of the present invention is not particularly limited. In some embodiments, the composition according to the present invention is preferably administered to the affected area several times at regular intervals (for example, 2 to 6 weeks or 3 to 5 weeks) to maintain an excellent effect. In other words, the composition can be administered repeatedly. The frequency of administration could be adjusted according to the subjects hair condition and the desired treatment outcome.

In some further embodiments, the method of the present disclosure further comprises administering a second active agent, wherein the second active agent is used with the composition of the present invention sequentially, concurrently or separately.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

The present disclosure can be more fully understood by reading the following descriptions of the embodiments, with reference made to the accompanying drawings.

FIG. 1 is a figure showing the effect of the treatment of BB101 or TMD23 on cell viability.

FIG. 2 is a photograph showing the effect of BB101 or TMD23 versus Finasteride on hair growth in mice at day 1 to day 11 after administration.

FIG. 3 is a photograph showing the effect of BB101 or TMD23 versus Finasteride on hair growth in mice at day 13 to day 17 after administration.

### DETAILED DESCRIPTION OF THE INVENTION

The technical solutions illustrated in the examples of the present disclosure will now be described more clearly and completely, and it will be apparent that the described examples are merely part of the examples of the present disclosure and are not intended to be exhaustive. The present disclosure can also be implemented or applied as described in different examples. All other examples obtained without creative work by those skilled in the art are within the scope of the present disclosure.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “comprising,” “comprises,” “include,” “including,” “have,” “having,” “contain,” “containing.” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the term “preventing” or “prevention” refers to preventive or avoidance measures for a disease, symptoms or conditions, which include, but are not limited to, applying or administering one or more active agents to a subject who has not yet been diagnosed as a patient suffering from the disease, symptoms or conditions but may be susceptible or prone to the disease, symptoms or conditions. The preventive measures of the present disclosure are provided to avoid, prevent, or postpone the occurrence of the disease, symptoms or conditions. e.g., hair loss.

As used herein, the term “treating” or “treatment” refers to obtaining a desired pharmacologic, physiologic, and/or cosmetic effect, e.g., restoration of hair growth. The effect may be prophylactic in terms of completely or partially preventing a condition, appearance, disease or symptom and/or may be therapeutic in terms of a partial or complete cure for a condition and/or adverse effect attributable to a condition or disease.

As used herein, the term “administering” or “administration” refers to the placement of an active agent into a subject by a method or route which results in at least partial localization of the active agent at a desired site to produce a desired effect. The active agent described herein may be administered by any appropriate route known in the art. For example, the composition of the present disclosure is administered to the subject by intradermal administration.

As used herein, the term “an effective amount” means a sufficient amount that the composition can provide a hair growth promoting effect in a reasonable beneficial-harmful ratio. Within appropriate medical judgment, the amount and proportion of active ingredient is the condition of the affected area, the degree of symptoms, the cause of the disease, the duration of treatment, the specific active ingredient contained, the concentration, the administration method utilized, and the general health of the patient. It can vary depending on factors such as the condition, the subject's tolerance to the administration, other drugs administered to the subject.

As used herein, the term “recombinant” may refer to the alteration of genetic material by human intervention. For example, recombinants may refer to the manipulation of DNA or RNA in a cell or virus or an expression vector by molecular biology (recombinant DNA technology) methods, including cloning and recombination. Recombinant may also refer to the manipulation of DNA or RNA in a cell or virus by random or directed mutagenesis. A “recombinant” nucleic acid can be described with reference to how it differs from a naturally occurring counterpart (the “wild-type”). A recombinant protein may refer to a protein expressed by recombinant DNA technology. A recombinant protein has a similar amino acid sequence and maintains the same activity or function as its parental protein.

As used herein, the term “intradermal” means within the dermis tissue of the skin, and “subcutaneous” means administering the composition to the adipose tissue of the skin.

The numeral ranges used herein are inclusive and combinable, any numeral value that falls within the numeral scope herein could be taken as a maximum or minimum value to derive the sub-ranges therefrom. For example, it should be understood that the numeral range “0.05 μg to 1 mg” comprises any sub-ranges between the minimum value of 0.05 μg to the maximum value of 1 mg, such as the sub-ranges from 0.05 μg to 0.1 μg, 0.3 μg to 5 μg. 1 μg to 10 μg, 15 μg to 500 μg, from 10 μg to 1000 μg, from 30 μg to 80 μg and so on. In addition, a plurality of numeral values used herein can be optionally selected as maximum and minimum values to derive numerical ranges. For instance, the numerical ranges of 0.1 μg to 50 μg, 0.1 μg to 100 μg, and 50 μg to 100 μg can be derived from the numeral values of 0.1 μg. 50 μg, and 100 μg.

As used herein, the term “about” generally refers to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

As used herein, the term “hair” in the present disclosure is not limited to the hair that grows on the scalp, but includes eyelashes, eyebrows, beards, ears, nose, chest, pubic hair, etc., and means any hair on the scalp, head, face, and body.

As used herein, the term “hair growth” is to induce the growth of a new hair cycle, prolong the anagen phase, increase the rate of hair growth and thicken the hair.

As used herein, the term “hair loss” and “thinning of hair” mean hair density loss, shortened hair growth phase, reduced hair shaft thickness, and reduced number of hairs, which may be aging, genetic causes, or others. It can be evoked by the cause of and can correspond to both men and women of all ages.

As used herein, the terms “patient” and “subject” are used interchangeably. The term “subject” means a human or other animals. Examples of the subject include, but are not limited to, a human, monkey, mouse, rat, woodchuck, ferret, rabbit, hamster, cow, horse, pig, deer, dog, cat, fox, wolf, chicken, emu, and ostrich. In at least one embodiment of the present disclosure, the subject is a mammal, e.g., a primate such as a human.

As used herein, the term “nucleic acid molecule” refers to a deoxyribonucleotide or ribonucleotide sequence in a double-stranded or single-stranded form, and generally includes naturally occurring nucleotides or artificial chemical mimics, of which the sources are not limited herein. The term “nucleic acid” as used herein includes natural or unnatural gene, polynucleotide, oligonucleotide, RNA, mRNA. DNA, cDNA, or nucleic acid analogue, but the present disclosure is not limited thereto.

As used herein, the term “nucleic acid analogue” refers to compounds or artificial nucleic acids which are structurally or functionally equivalent to natural RNA or DNA. In some embodiments, the nucleic acid analogue may have at least one part of a nucleotide (e.g., the nucleobase, the phosphate backbone, or the pentose sugar) being modified to alter the structure of the nucleic acid.

Many examples have been used to illustrate the present disclosure. The examples below should not be taken as a limit to the scope of the present disclosure.

### EXAMPLES

For a more detailed description of the present disclosure, the method disclosed will be provided and described in detail with reference to the following examples. The materials used in the present disclosure but unannotated herein are commercially available.

### Example 1: Effect of TMD23 and BB101 on Cell Viability

1×10⁴ hair follicle dermal papilla cells (HFDPC) per well were cultured in the 96-well plate in 12 hours and were exposed to 1 mM testosterone. The cells were then treated with different concentrations of BB101 and TMD23. After 24 hours of treatment, cell viability was examined by MTT and presented as a percentage of DMSO control.

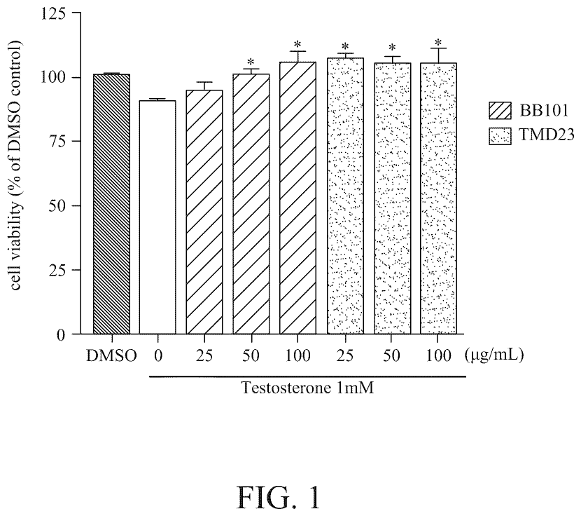

As shown in FIG. 1, the results indicated that treatment of either BB101 or TMD23 significantly increases cell viability as compared to the control group.

### Example 2: TMD23 Accelerated Hair Growth in Mice

Six-week-old male C57BL/6 mice were purchased from National Laboratory Animal Center. Space allocation for 10 animals was 29×18×13 cm³. All animals were maintained in a controlled temperature (22° C.-24° C.) and humidity (50%-60%) environment with 12-hour light dark cycles for at least one week. Free access to standard lab chow for mice and RO water was granted. All aspects of this work including housing, experimentation and disposal of animals were performed in accordance with the Guide for the Care and Use of Laboratory Animals (National Academy Press, Washington. D. C., 1996).

The animals were housed and allowed to adapt to the laboratory environment for 1 week, after which the 7-week-old mice were divided into five groups of 3 mice each: a 50 μg/mL of BB101 treatment group, a 200 μg/mL of BB101 treatment group, a 50 μg/mL of TMD23 treatment group, and a 200 μg/mL of TMD23 treatment group.

To assess the effects of hair growth, the back of each mouse was shaved 1 day before the beginning of the experiment, using an electric clipper for animals. The back was divided into two areas, both areas were topically applied testosterone once a day throughout the experiment onto the shaved skin of each mouse. The lower part was topically applied the test drug TMD23, BB101, and Finasteride through the transdermal drug delivery system, derma stamp, on to the shaved skin once a day, 2 stamps for each administration (the volume was about 20 μL). The upper part was given water by the drug delivery system as the solution control group. The mice were photographed with a digital camera under ether anesthesia, to evaluate the darkening of skin color on day 1, day 9, day 11, day 13, day 15, and day 17. The animal care and the protocol for this study were in accordance with IACUC (Institutional Animal Care and Use CommitteeLAC-2022-0137) guidelines.

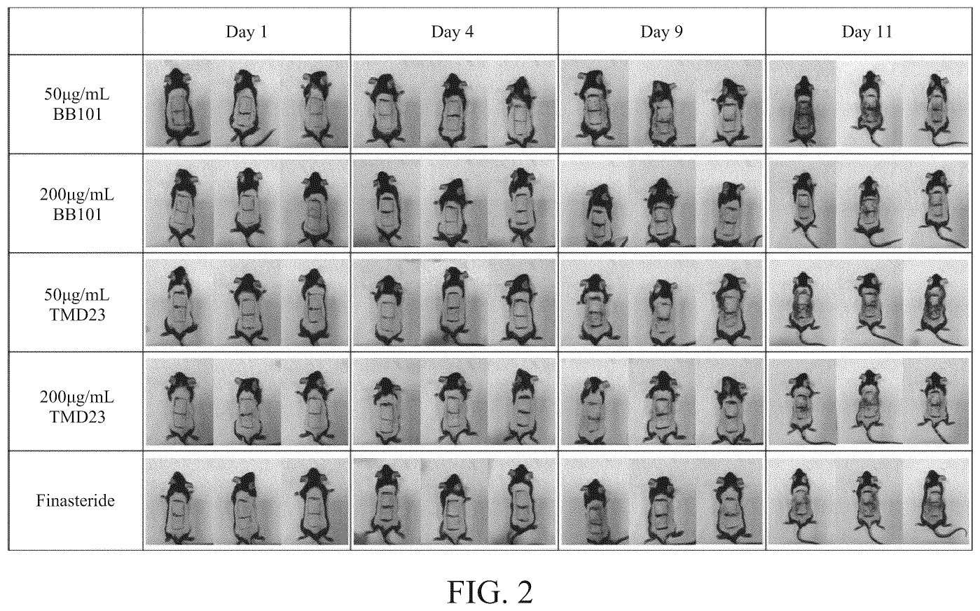

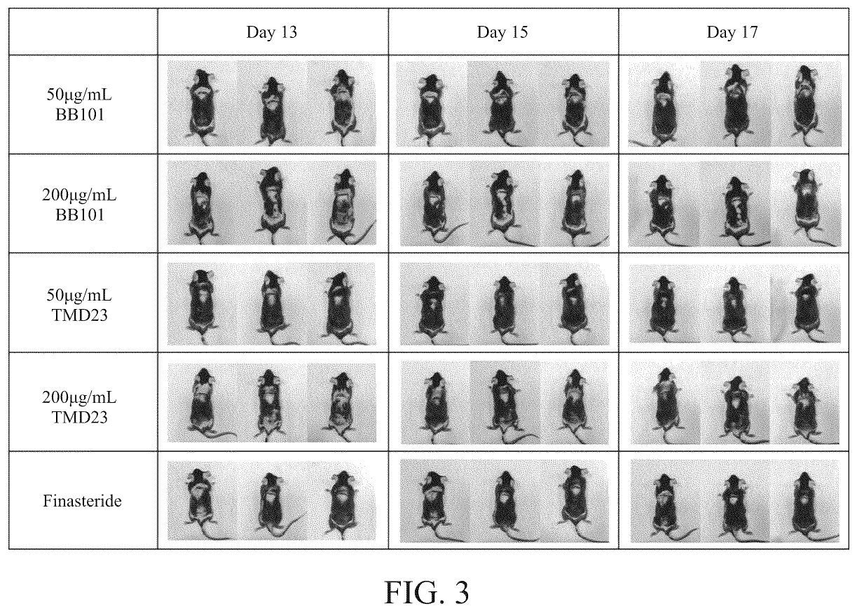

The results showed that at day 17 after administration, the darkening of skin color between Test groups and Positive (Finasteride) treatment groups were comparable, indicating that TMD23 and BB101 are effective in accelerating hair growth and treating hair loss (FIG. 2 and FIG. 3).

While some of the embodiments of the present disclosure have been described in detail above, it is, however, possible for those of ordinary skill in the art to make various modifications and changes to the embodiments shown without substantially departing from the teaching and advantages of the present disclosure. Such modifications and changes are thus encompassed in the scope of the present disclosure as set forth in the appended claims.

What is claimed is:

1. A method for preventing or treating hair loss or facilitating hair growth or regrowth, comprising administering a composition to a subject in need thereof, wherein the composition comprises an effective amount of a polypeptide or a nucleic acid molecule encoding the polypeptide, and the polypeptide comprises an amino acid sequence having an EGF-like domain of thrombomodulin or a conservative variant thereof.

2. The method of claim 1, wherein the polypeptide is a recombinant polypeptide comprising an amino acid sequence of EGF-like structures of the thrombomodulin operably linked to a serine-threonine rich domain of the thrombomodulin or the conservative variant thereof.

3. The method of claim 2, wherein a number of the EGF-like structures is one to six.

4. The method of claim 3, wherein therecombinant polypeptide essentially consists of an amino acid sequence of six EGF-like structures of the thrombomodulin operably linked to the serine-threonine rich domain of the thrombomodulin or the conservative variant thereof.

5. The method of claim 1, wherein the polypeptide is at least 35% identical to TMD23 having the amino acid sequence of SEQ ID NO: 1.

6. The method of claim 1, wherein the polypeptide essentially consists of BB101 having the amino acid sequence of SEQ ID NO: 2.

7. The method of claim 1, wherein the polypeptide comprises a TME5 structure having the amino acid sequence at least 35% identical to SEQ ID NO: 3.

8. The method of claim 1, wherein the polypeptide comprises a TME5C structure having the amino acid sequence at least 35% identical to SEQ ID NO: 4.

9. The method of claim 1, wherein the nucleic acid molecule is an exogenously introduced polynucleic acid for increasing the expression of the polypeptide in a subject.

10. The method of claim 1, wherein the hair loss is selected from the group consisting of baldness, involutional alopecia, androgenic alopecia, male-pattern hair loss, female-pattern hair loss, alopecia areata, traction alopecia, anagen effluvium, telogen effluvium, cicatricial (scarring) alopecia, hair loss due to infectious diseases, hair loss due to chemotherapy, hair loss due to radiation, hair loss due to hormonal imbalance, hair loss due to medication, hair loss due to chemicals exposure, hair loss due to stress, and any combination thereof.

11. The method of claim 1, wherein the composition is administered to the skin of the subject.

12. The method of claim 1, wherein the composition is administered to the subject topically, intradermally, or subcutaneously.

13. The method of claim 1, further comprising applying physical intervention, chemical intervention, or a combination thereof.

14. The method of claim 13, the physical intervention is selected from the group consisting of needles, microneedles, microneedle patches, micro-needling rollers, micro-needling stamps, Meso Gun, microsurgical device, shave, superficial injection device, needle-free intradermal injection, temperature elevation, electroporation, ultrasound-mediation, ultrasound-mediated microbubble cavitation, and any combination thereof.

15. The method of claim 1, wherein the composition further comprises a pharmaceutically or cosmetically acceptable carrier.

16. The method of claim 1, wherein the composition is formulated in a form selected from the group consisting of a gel, cream, paste, lotion, spray, suspension, solution, dispersion salve, hydrogel, liquid, emulsion, ointment, a film, an orabase, a powder, and liniment.
